(19) United States
(12) Patent Application Publication
(10) Pub. No.: US 2018/0137621 A1
(43) Pub. Date: May 17, 2018

(54) A Method of Analysing an Image for Assessing a Condition of an Organ of a Patient

(71) Applicants: Telethon Kids Institute, Subiaco, Western Australia (AU); Erasmus University Medical Center Rotterdam, Rotterdam (NL)

(72) Inventors: Stephen Stick, Subiaco, Western Australia (AU); Tim Rosenow, Subiaco, Western Australia (AU); Harmannus Arnoldus Wilhelmus Maria Tiddens, Rotterdam (NL); Marleen De Bruijne, Rotterdam (NL)

(73) Assignees: Telethon Kids Institute, Subiaco, Western Australia (AU); Erasmus University Medical Center Rotterdam, Rotterdam (NL)

(21) Appl. No.: 15/574,247

(22) PCT Filed: Mar. 9, 2016
(86) PCT No.: PCT/AU2016/000079

§ 371 Date: Nov. 15, 2017

(30) Foreign Application Priority Data
May 20, 2015 (AU) 2015901841

(51) Int. Cl. G06T 7/00; G06T 7/11

(52) U.S. Cl. CPC G06T 7/0016; G06T 7/11; G06T 2207/10081; A61B 6/5217; G06T 2207/10012; G06T 2207/30061; G06T 2207/10088

(57) ABSTRACT

A method is disclosed for analysing an image to assess a condition of an organ of a patient represented in the image. The method includes initially selecting a spatial resolution for an inspection matrix having a number of inspection regions each delimiting a part of the image. The inspection matrix is applied to the image and the part of the image within each of the inspection regions is analysed to determine a condition thereof. The condition is compared with a predetermined criterion and, if the condition is deemed to satisfy the criterion, the inspection region is identified, e.g. by annotating it with a colour. Finally a quantitative measure of an extent of the condition in the image is provided that is based on a number of the identified or annotated inspection regions.

### FIELD OF THE INVENTION

The present invention relates to a method of analysing an image for assessing a condition of an organ of a patient. By way of example only, the method may be used to assess the extent of symptoms of cystic fibrosis in a lung of a patient.

### BACKGROUND

Standardised outcome measures are in one sense a measuring tool for diagnosis, monitoring disease progress, or to evaluate the effectiveness of certain treatments in medical research. For example, standardised outcome measures are used in clinical trials to test the effectiveness of a certain drug being trialled on a patient over a period of time. It is important that standardised outcome measures are repeatable and can produce reliable results.

Currently there are no standardised outcome measures appropriate for very young children with cystic fibrosis. This effectively excludes infants and young children from clinical trials in an era where new, potentially disease modifying drugs, are becoming available. Structural lung disease or “airways disease” in cystic fibrosis begins early in life, is progressive, and is often the only evidence of respiratory disease in children less than 6 years of age.

Several computed tomography (CT) scoring systems for cystic fibrosis have been developed for adults and children over the age of 6. However, these methodologies are semi-quantitative, and are not appropriate for assessing subtle appearances and low extents of structural changes found early in life.

### SUMMARY OF THE INVENTION

The present invention seeks to provide a method of assessing the extent of a condition in an image of an organ or other body part of a patient, which may be particularly useful in addressing the above mentioned problems.

In one aspect the present invention provides a method of analysing an image for assessing a condition of an organ of a patient represented in the image, the method comprising the steps of:
selecting a spatial resolution for an inspection matrix comprising a number of inspection regions each delimiting a part of the image;
applying the inspection matrix to the image;
analysing the part of the image within each of the inspection regions to determine a condition thereof;
comparing the condition with a predetermined criterion;
identifying the inspection regions for which the condition is deemed to satisfy the criterion; and
providing a quantitative measure of an extent of the condition in the image based on a number of the identified inspection regions.

The inspection matrix may be a two-dimensional matrix that may be delimiting a number of pixels in the image.

The spatial resolution of the inspection matrix may be dependent on a size of the organ represented in the image or an age of the patient from whom the image of the organ is taken. The size of the inspection regions of the inspection matrix that is selected for a patient and a specific organ may be selected proportional to a size of the organ (and/or the size and/or the age of the patient). For example, the spatial resolution may be higher for an organ of child than for an organ of an adult such that a comparable number of inspection regions are used for both the organ of the child and the organ of the adult. This provides the opportunity to quantify an extent of the disease for the child in the same manner as for the adult. In other words, by providing that the size of the inspection regions is based on the size of the organ or age of the patient, the present invention may provide the advantage of an accurate and sensitive assessment tool for monitoring disease progress and for clinical trials and that is largely independent from age of a patient. It may provide the further advantage of a quantitative measure that is sensitive to early structural lung disease for use in clinical trials or longitudinal assessment, particularly for children under 6 years of age.

In one specific embodiment of the present invention the image for assessing a condition of an organ of a patient represented in the image is one of a plurality of images and the condition of the same organ is assessed for each patient of a plurality of patients, wherein the step of selecting the spatial resolution for the inspection matrix is conducted for each patient and such that the spatial resolutions of the inspection matrices decreases substantially linearly with an increase in a size of the organs of the patients. The step of selecting the spatial resolution may comprise selecting the spatial resolution such that, largely independent from age of a patient and size of the organ, the inspection matrices have substantially the same, or at least similar, number of inspection regions.

The spatial resolution may be selected so that each inspection region has a dimension being 1%, 2%, 3%, 4%, 5% or less of an overall dimension of the organ represented in the image.

The image is typically a cross-sectional image and the dimension may be a cross-sectional width dimension.

The condition may be deemed to satisfy the criterion if the condition is prevalent over an area covering more than 10, 20, 30, 40, 50, 60, 70, 80 or 90% of the inspection region.

The condition may be deemed not to satisfy the criterion if the condition is prevalent over an area covering more than 10, 20, 30, 40, 50, 60, 70, 80 or 90% of the inspection region.

The step of identifying the inspection regions may comprise annotating the respective inspection regions with a colour.

The step of providing the quantitative measure may comprise providing a proportion value of the number of identified inspection regions within the inspection matrix.

The step of providing the quantitative measure may also comprise counting a number of identified inspection regions and dividing this number by a total number of inspection regions in the inspection matrix.

The image may be one of: a computed tomography (CT) image, a radiograph image, and an MRI image.

The image may comprise an inspiratory scan of the patient.

The image may comprise an expiratory scan of the patient.

The criterion may comprise the presence of a disease in the organ represented in the image.

The method may comprise establishing a marker of the disease if the quantitative measure exceeds a predetermined value.

The marker may be one of: bronchiectasis; mucous plugging; bronchial wall thickening, atelectasis, or another lung disease.

The marker may be analysed and determined in a hierarchical manner in the order of: bronchiectasis; mucous plugging; bronchial wall thickening, atelectasis, or another lung disease.

The method may be repeated at one or more distinct time intervals such that a change in the disease can be determined.

The method may be repeated on a plurality of related images, wherein the plurality comprises between one and five, six and fifteen, or approximately ten images of the organ.

The related images may be equidistant two-dimensional slices taken through a three-dimensional image of the organ.

The image may comprise a cross-sectional representation of a lung.

The condition may be cystic fibrosis.

The image may comprise an organ of a patient having an age of 10, 9, 8, 7, 6, 5, 4, 3, 2 or 1 years.

The steps of analysing the part of the image, comparing the condition thereof and identifying the inspection regions may be all conducted autonomously by a computer system.

In another aspect the present invention provides a method of assessing the extent of a condition in a body part of a patient, the method comprising:
obtaining at least one cross-sectional image of the body part of the patient;
selecting a size for an inspection region of the cross-sectional image suitable for determining a criterion of the condition based on a size of the body part or an age of the patient;
dividing the cross-sectional image into a plurality of the inspection regions;
analysing one or more of the inspection regions to determine whether a criterion of the condition is satisfied;
identifying the inspection regions for which the criterion is satisfied; and
providing a quantitative measure of the extent of the condition in the body part based on the identified inspection regions.

Dividing the representation into a plurality of regions may comprise superimposing a grid on the representation. The grid may have cells that have a size that corresponds to that of the selected inspection region. Each grid cell may have a width that is 1, 2, 3, 4, 5% or less of the width of the body part. The method may comprise repeating the method after a period of time such that a progression or treatment of the disease can be monitored.

In a further aspect the method may follow a fully automatic, data-driven approach for texture-based quantitative analysis of the condition in the image.

### BRIEF DESCRIPTION OF THE DRAWINGS

Notwithstanding any other forms which may fall within the scope of the method as set forth in the Summary, specific embodiments will now be described, by way of example only, with reference to the accompanying drawings in which:

FIG. 1 is a flow chart depicting the method according to an embodiment of the present invention;

FIG. 2 is a cross-sectional image representing an organ or body part;

FIG. 3a depicts an uppermost cross-sectional image to be used according to the method;

FIG. 3b depicts a lowermost cross-sectional image to be used according to the method;

FIG. 4a depicts an unannotated inspiratory cross-sectional image of a lung;

FIG. 4b depicts an annotated inspiratory cross-sectional image of a lung;

FIG. 5a depicts an unannotated expiratory cross-sectional image of a lung;

FIG. 5b depicts an annotated expiratory cross-sectional image of a lung.

### DETAILED DESCRIPTION

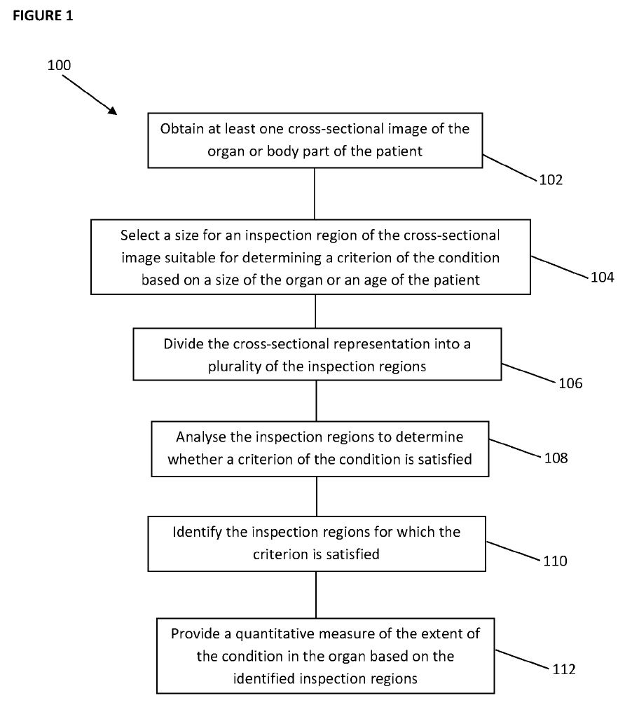

FIG. 1 illustrates a method 100 of assessing the extent of a condition in an organ or body part of a patient. In a specific embodiment, which will be described herein, the condition is cystic fibrosis and the organ is a lung. However, the method 100 is not limited to this embodiment, and may be used for assessing other conditions, such as disorders causing immune deficiency, primary ciliary dyskinesia, and non-cystic fibrosis bronchiectasis, or diseases in other organs and body parts.

The method 100 may be used as a measuring tool for assisting in the diagnosis, monitoring the progression of cystic fibrosis, or to evaluate the effectiveness of drugs in clinical trials. For example, the method may be repeated after a period of time and at discrete time intervals such that a progression or treatment of the disease can be monitored. Thus, the method may be used to identity or establish a marker or indicator associated with the condition to assist in determining whether or not the patient has the condition, and if so, the extent thereof. The method may also use a known marker of a disease as a criterion in the assessment.

Although the method 100 may be suitable for any patient, the method 100 seeks to provide the particular advantage of being suitable and effective for assessing young patients. This is because young patients generally have smaller body parts and may not exhibit as many symptoms of a disease compared to adult patients. Thus the disease may be more difficult to detect, and requires the assistance of a more sensitive method of detection or assessment such as provided by the present disclosure. More specifically, it is believed that the method 100 is particularly suitable for patients under six years of age with cystic fibrosis or other structural lung disease. As previously mentioned, known methods are not suitable for detecting subtle symptoms of cystic fibrosis found early in life.

To provide a general idea, the method 100 comprises:
obtaining at least one cross-sectional image of a body part of a patient (step 102);
selecting a size for an inspection region of the cross-sectional image suitable for determining a criterion of the condition based on a size of the body part or an age of the patient (step 104);
dividing the cross-sectional image into a plurality of inspection regions (step 106);
analysing one or more of the inspection regions to determine whether a criterion of the condition is satisfied (step 108);
identifying the inspection regions for which the criterion is satisfied (step 110); and
providing a quantitative measure of the extent of the condition in the body part based on the identified regions (step 112).

Further, the method 100 according to the specific embodiment herein described is carried out according to two general aspects: obtaining inspiratory (breathing in) and expiratory (breathing out) chest computed tomography (CT) scans. In general, the inspiratory scans are assessed for the presence of airways disease, and the expiratory scans are assessed for the trapped air. These are also known in the art as “markers” of cystic fibrosis. Each step of the method 100 will now be described in more detail.

### Step 102

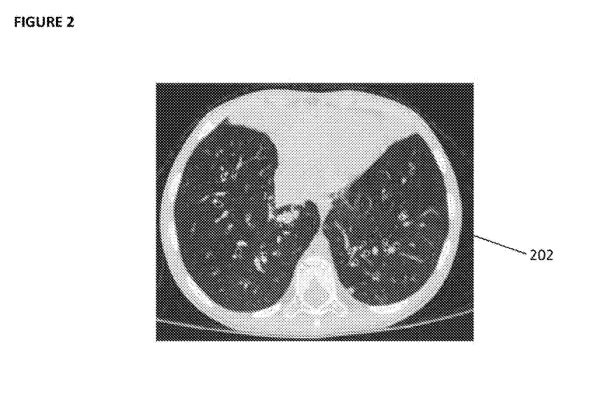

Step 102 comprises obtaining at least one cross-sectional image or representation of the body part of the patient. With reference to FIG. 2, the cross-sectional image according to the specific embodiment herein described is a two-dimensional image or a “slice” 202 of a chest CT scan of a patient. By way of background, a CT scan itself involves using X-rays to produce tomographic images (or sections) of a scanned object. Thus, CT scans are commonly used in medical imaging because it can provide a view of an inside of an object without cutting. The slices are then commonly used to generate a three-dimensional image. However, a person skilled in the art will appreciate that other forms of medical imaging may be suitable, such as magnetic resonance imaging (MRI) or radiograph imaging, to generate the three-dimensional image.

Volumetric inspiratory and expiratory CT scans of the patient are taken. Once a CT scan of the patient has been taken, the scan is converted to an image of a lung on a user interface or computer, utilising software such as MIPAV (Medical Image Processing, Analysis, and Visualization). The software is one that enables quantitative analysis and visualization of medical images.

For inspiratory scans, the thinnest slice 202 reconstruction is used, for example, 0.8 mm-1.0 mm. For expiratory scans, a slice thickness of 4 mm-5 mm and the smallest slice spacing possible is used. A minimum intensity projection is also used for the expiratory scans, which increases visibility of low intensity regions and facilitates visualisation of trapped air.

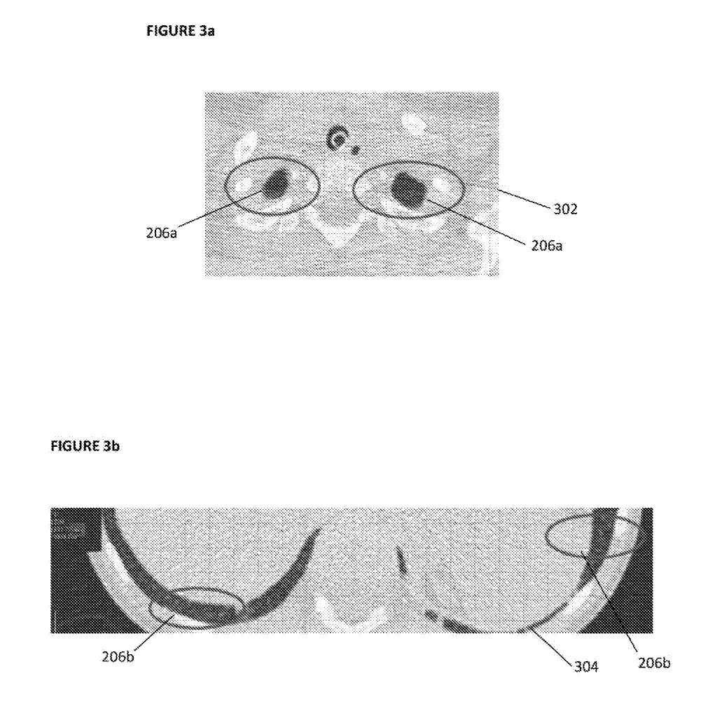

In this specific embodiment, ten equidistant axial slices from each inspiratory and expiratory scan are obtained to be analysed according to the method 100. The slices are obtained generally between the apex and the base of the lung. This can be done using SALDSegVol grid software package. Specifically, with reference to FIGS. 3a and 3b, the slices are obtained by:
locating the uppermost slice 302 in which, for both lungs, at least 50% of a grid cell 206a (which will be explained in more detail under “Steps 104 and 106”) contains a portion of a lung, and noting the number of the uppermost slice;
locating the lowermost slice 304 in which for both lungs, at least 50% of a grid cell 206b contains a portion of a lung, and doing the same; and
dividing the difference between the uppermost slice number and lowermost slice number by 11, to provide a spacing between each slice 202 (or “slice interval”) with which to obtain or identify the ten slices to be analysed.

The ten slices 202 obtained by the above process may be identified to the observer by the grid software indicating which slices should and should not be analysed.

It is noted that during experimental testing, it was found that slices with 5 mm and 10 mm, and 5 mm and 20 mm intervals exceeded a 0.99 intra-class correlation coefficient for all outcome measures, and ten slices ensures that the intervals are less than 20 mm for the under six years age group. Specifically, this was done by assessing slices 202 annotated at 5 mm intervals and analysing the slices 202 according to the method 100. Then, every second annotated slice 202 is removed to provide 10 mm intervals, and then again to provide 20 mm intervals. The maximum suitable distance between annotated slices was determined by finding the largest interval that did not result in a significant change in the outcomes of the method 100. Based on these results, a fixed number of ten annotated slices was chosen as a suitable standard. This standard was used for assessing all other data sets, and takes approximately 20 minutes to analyse. However, a person skilled in the art will understand that the number of slices may be suitably varied.

### Steps 104 and 106

Steps 104 and 106 may be considered as going hand-in-hand, and therefore will be described together.

Step 104 comprises selecting a size of an inspection region of the cross-sectional image suitable for determining a criterion of the condition based on a size of the body part or an age of the patient. With reference to “Step 102” above, it will be appreciated that the “inspection region” in this embodiment corresponds to a grid cell 206. As previously mentioned, in this embodiment the condition is the disease of cystic fibrosis and further, the criterion relates to the presence of cystic fibrosis in lungs.

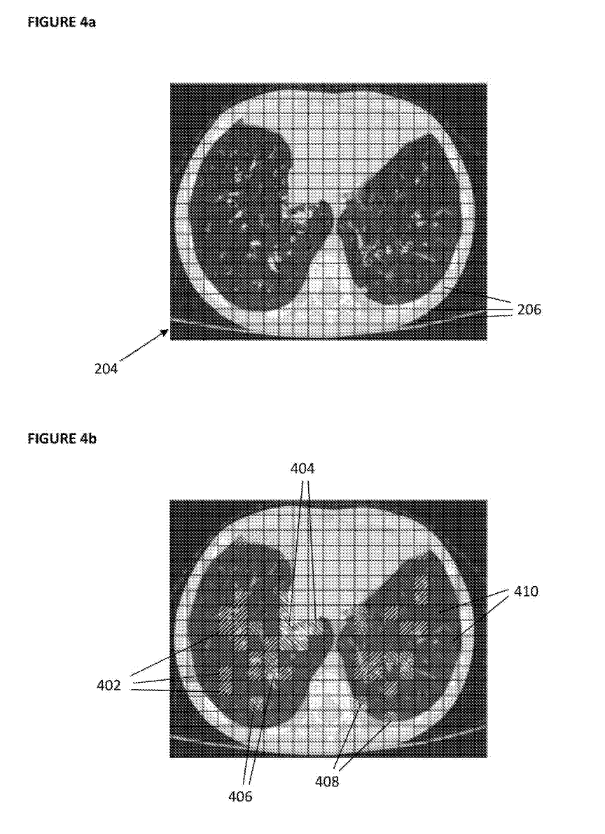

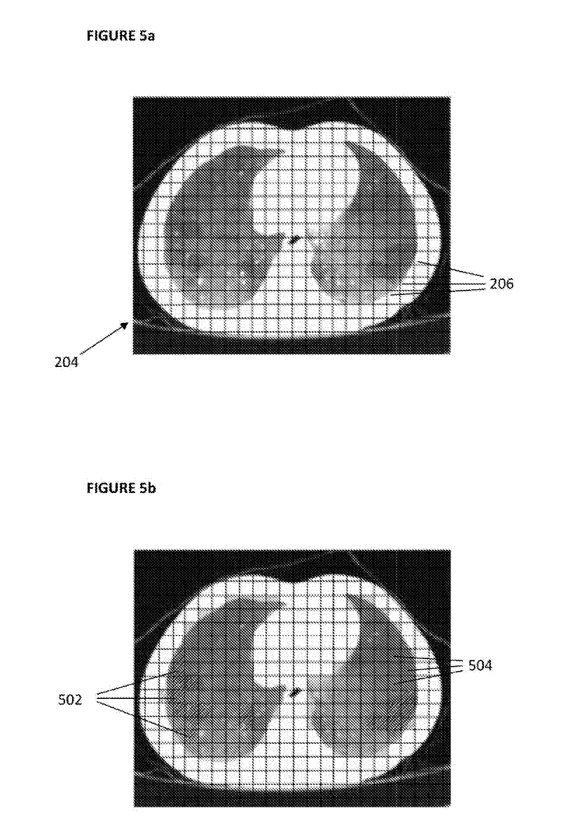

Step 106 then comprises dividing the cross-sectional image or slice 202 into a plurality of the inspection regions or cells 206. Thus, in this embodiment, step 106 involves formulating the grid 204 of grid cells 206. More specifically, with reference to FIGS. 4a-5b, in this embodiment, the step 106 involves superimposing or overlaying a grid 204 onto the image of the slice(s) 202 of both expiratory and inspiratory scans, using the grid software. The grid software may allow for one grid to be applied to all slices obtained from the CT scan. Thus, the regions in the step 104 are in the form of grid cells 206 of the grid 204. However, first the size of the grid cells is to be selected, in accordance with step 104.

Each grid cell 206 is a square, the size of which is determined according to the size of the particular lungs to be analysed. In this specific embodiment, according to step 104, the size of each cell 206 (or “grid size”) may be selected by:
Selecting a slice 202 of an anatomical reference or landmark, such as the first slice after the bifurcation of the trachea (otherwise known as the slice closest to the carina). The carina was chosen because it represents a stable landmark that represents the approximate lung size across patients. However, it will be appreciated that any suitable reference or landmark can be used, particularly one that can be reliably and repeatedly identified if multiple tests are taken over a period of time with the same patient and/or across a number of patients.
Measuring the horizontal distance between the left-most and right-most extent of the lung field.
Dividing the horizontal distance by 20, rounded to the nearest millimetre.

For example, a width at the carina may be 151 mm, which corresponds to a grid size of 151/20=7.55=8 mm. This size may be set using the grid software and/or electronic callipers using Myrian software (Intrasense, Montpellier, France) or another suitable radiological software. It is noted that this grid cell size was arbitrarily chosen to be 1/20th of the lung width. This size was selected as it approximately represents the size of the largest assessable airway in the lung.

However, it will be appreciated that other grid cell sizes may also be suitable, for example, each grid cell may have a width that is between 5% and 10% of the width of the body part, or less than 5%. The grid cells 206 may also be rectangular.

Also, as previously mentioned, the ten slices obtained in step 102 are in one embodiment obtained by utilising the grid cells 206. Therefore, a person skilled in the art will understand that the steps 102 and 104 (and step 106) are not necessarily required to be carried out in a strict order.

### Steps 108 and 110

Steps 108 and 110 also may be considered as going hand-in-hand, and therefore will be described together.

The step 108 more particularly involves analysing one or more of the inspection regions or grid cells 206 to determine whether a criterion of the condition is satisfied. More specifically, this step involves analysing to determine whether the lung portion defined by the grid cell 206 satisfies a predetermined criterion associated with cystic fibrosis.

The step 110 further involves identifying the regions or grid cells 206 for which the criterion is satisfied. In this particular embodiment, identifying the cell also comprises annotating the cell according to whether or not it has met the predetermined criterion.

In the specific embodiment herein described, in step 108, grid cells 206 will only be identified for analysis and annotated if at least 50% of the cell includes a portion of the lung under examination (as opposed to any other matter). Cells containing less than 50% lung are thus left unannotated.

For inspiratory scans, the criterion against which suitable grid cells 206 are analysed according to step 108, is whether the defined lung portion shows a marker, such as the presence of bronchiectasis, mucous plugging or other airway abnormalities. Prior to analysis, the slice overlaid with the grid 204 is depicted in FIG. 4a. Then, with reference to FIG. 4b, if a grid cell 206 is identified as showing an airway abnormality, the grid cell 206 is annotated using unique indicia representing that abnormality, according to step 110. Annotation in this embodiment is done by grid cell 206 colouring. For example, the following may be applied:

TABLE 1

| Any part of cell having this criteria | Cell colour | Reference |
|---|---|---|
| Bronchiectasis | Red | 402 |
| Mucous plugging or consolidation | Yellow | 404 |
| Otherwise abnormal airway, e.g. | Orange | 406 |
| bronchial wall thickening, “saccular” | | |
| appearance | | |
| Atelectasis (partial collapse) that is | Magenta | 408 |
| likely not pathological, e.g. caused | | |
| by anaesthesia | | |
| Healthy tissue | Green | 410 |

In the analysis, bronchiectasis may be identified by visual inspection of whether the outer edge bronchus-artery cross-sectional area ratio is greater than one. Mucous plugging may be identified by high density airway occlusion or tree-in-bud appearance. Bronchial wall thickening may be identified by airway walls that appear thicker or have increased signal intensity relative to normal airways.

The grid cells 206 containing atelectasis are to be excluded from all analysis as they are likely related to general anaesthesia rather than pathology. Thus, the grid cells 206 annotated with bronchiectasis, mucous plugging or an otherwise abnormal airway are also known as the ‘assessable cells’.

The above annotation is done according to a hierarchical system as indicated in Table 1 from highest to lowest priority. In other words, bronchiectasis has a higher priority than mucous plugging, which in turn has higher priority than bronchial wall thickening, and so forth.

For expiratory scans, the criterion against which suitable grid cells 206 are assessed according to step 108, is the amount of trapped air in the part of the lung slice defined by the cell. Again, prior to annotation, the slice 202 for an expiratory scan slice overlaid with a grid 204 is depicted in FIG. 5a. Then, in carrying out step 110, the cells are annotated according to whether trapped air represented 50% or more of the lung part defined by the cell (trapped air) or less than 50% (healthy). Thus, with reference to FIG. 5b, the following may be applied:

TABLE 2

| Cell has this criteria | Cell colour | Reference |
|---|---|---|
| More than 50% of the lung part contains | Blue | 502 |
| trapped air | | |
| At least 50% of the lung part does not | Green | 504 |
| contain trapped air | | |

The annotation may be done using the grid software previously mentioned, by clicking on a cell and applying a suitable colour.

Although only select slices 202 were annotated, using the software programs herein described or other suitable programs, it is possible for the observer to scroll through the entire lung volume provided by the CT scan to assist in classification. For example, scrolling can aid in distinguishing between an occluded airway and an artery.

In conducting the above analysis, further criterion associated with how much of the lung portion defined by grid cell 206 is affected by the condition, may also be applied. For example, the criterion may be deemed satisfied if an area that covers more than a certain percentage of a cell 206 is indicative of the disease. The opposite may also be applied, for example, the criterion may not be satisfied if an area that covers more than a certain percentage of a cell 206 is indicative of the disease.

### Step 112

The step 112 comprises providing a quantitative measure of the extent of the condition in the body par: based on the identified regions. More specifically, the quantitative measure is provided based on the grid cells 206 annotated with cystic fibrosis markers. In this regard, the primary quantitative measures or outcomes obtained are:
For inspiratory scans, “% DIS” (the volume proportion of the lung with airways disease), and “% Bx” (the volume proportion of the lung with bronchiectasis); and
For expiratory scans, “% TA” (the volume proportion of the lung with trapped air).

The % DIS is determined by dividing the number of cells annotated with bronchiectasis, mucous plugging or airway abnormality by the total number of assessable cells, (i.e. annotated cells excluding atelectasis). For instance, with reference to Table 1 above, the following formula may be used:

$$\% DIS = 100 \times \frac{\# BE + \# Abnormal + \# Plug}{\# Total - \# Atelectasis}$$

where:
“BE”=red grid cell to indicate bronchiectasis
“Abnormal”=yellow grid cells to indicate mucous plugging
“Plug”=orange grid cells to indicate otherwise abnormal cell
“Total”=total number of cells annotated
“Atelectasis”=magenta grid cells to indicate atelectasis

The % Bx is determined by dividing the number of cells annotated with bronchiectasis by the total number of assessable cells, for instance:

$$\% Bx = 100 \times \frac{\# BE}{\# Total - \# Atelectasis}$$

The % TA is determined by dividing the number of cells with trapped air by the total number of cells annotated and expressing as a percentage. For instance, with reference to Table 2 above, the following formula may be applied:

$$\% TA = 100 \times \frac{\# AT}{\# Total}$$

where:
“AT”=blue cell to indicate cell with more than 50% trapped air
“Total”=total number of cells annotated

It is also conceived that the step 112 may also go towards establishing a marker of the condition by providing the quantitative measure of the extent of the condition. For example, a certain % DIS, % Bx or % TA figure might be specifically indicative of a certain condition, for example, patients who are at-risk for more severe disease or for lung infection.

The method 100 may be repeated for different patients of the same or different ages. For each patient a size of an inspection region (or a resolution of an inspection matrix) is selected. In order to be able to compare the extent of the disease for patients of different ages and/or different organ sizes, the inspection regions are selected such that the inspection regions have a size that is approximately proportional to the organ size of each patient and consequently for each patient substantially the same number of inspection regions is analysed.

In one embodiment of the present invention the method 100 is conducted to monitor progression or regression of a disease for example during treatment. In this case the method 100 is repeated frequently and/or periodically (for example within a few weeks, months or years) and results of each analysis are compared with each other to provide information about the progression or regression of the disease.

In the above described embodiment, the method 100 is carried out manually by an operator working with a user-interface and performing the analysis of steps 102 to 112 by visual inspection of the slices 206. However, in another embodiment, the method 100 may be executed in an automated manner by a computer program, using for example textural analysis as described by U.S. Pat. No. 8,811,724. This may mitigate observer variability and increase efficiency.

In this regard, the automated computerised classification of the image of a lung or of a part of a lung comprises applying to the image under consideration a trained statistical classifier which has been trained by supervised learning on a training set of methodologically similar lung images each of which images has been previously manually annotated as described above to indicate the likelihood of the respective image relating to a lung characterised by a lung disease, such as bronchiectasis, mucous plugging, bronchial wall thickening, atelectasis, or another lung disease. During the training of the classifier, for each image in the training set the above described inspection matrix or grid is applied thereto to divide the image into the applicable inspection regions, and textural information relating to the intensities of locations within each inspection region obtained. Combinations of features of the textural information are used to suitably classify the training set inspection regions according to the annotated lung disease. In subsequently applying the trained statistical classifier to the image under consideration, in a computer a number of inspection regions are defined, and textural information relating to the intensities of locations within each inspection region of the kind used in training the classifier is obtained. Features of the textural information for the locations within the inspection regions of the image are combined as learnt in the training of the classifier to calculate probabilities of the inspection regions belonging to the specified lung disease, i.e. being bronchiectasis, mucous plugging, bronchial wall thickening, atelectasis, or another lung disease.

The term ‘methodologically similar’ implies that the images used in training the classifier and the image of interest that is to be classified should be of the same technical kind in order that they may meaningfully be compared. So a CT scan should be classified by the use of a model trained on CT scans, an MRI should be classified by the use of a model trained on MRIs and so forth. Preferably, the conditions under which the images used in training and the image of interest were obtained should all be as close as practical. Thus, in the case of CT scans, they should ideally be obtained using the same general kind or even the same model of scanner set up in the same way. However, some deviation from the exact same conditions will generally be acceptable, possibly with some degradation of the results.

A person skilled in the art will appreciate that the present invention was developed in the context of medical research. It is noted that in developing and testing the present invention, it was found that although the prevalence of structural lung disease early in life is high, the extent of the disease is relatively low, with a median proportion of lung volume affected being 0.87% and 1.86% at age 1 and 3, respectively. Thus, a sensitive and accurate quantitative method according to embodiments of the present invention is particularly advantageous for observing cystic fibrosis in early years. Moreover, it was found that test results obtained from carrying out the present method 100:
have high intra- and inter-observer agreement;
are better correlated to neutrophilic inflammation than traditional cystic fibrosis CT scoring methods; and
show stronger relationships between structural changes and trapped air progression.

Further still, it was found that % DIS at the age of 1 was significantly related to the change in % Bx over two years, suggesting that patients with worse baseline disease have faster progression of bronchiectasis. Therefore, the method 100 performed at the age of 1 year can potentially be used to identify patients at high risk for disease progression.

It is contemplated that the method 100 will be suitable for an intervention study with a relatively short duration, for example, two years. The present method 100 also provides the advantage of lending easily to ubiquitous access at medical centres to CT scanners, mean that multiple studies can be undertaken simultaneously around the world. This is an important consideration given the low incidence of cystic fibrosis.

Numerous variations and modifications will suggest themselves to persons skilled in the relevant art, in addition to those already described, without departing from the basic inventive concepts. All such variations and modifications are to be considered within the scope of the present invention, the nature of which is to be determined from the foregoing description.

In the description of the invention, except where the context requires otherwise due to express language or necessary implication, the words “comprise” or variations such as “comprises” or “comprising” are used in an inclusive sense, i.e. to specify the presence of the stated features, but no: to preclude the presence or addition of further features in various embodiments of the invention.

It is to be understood that, although prior art use and publications may be referred to herein, such reference does not constitute an admission that any of these form a part of the common general knowledge in the art, in Australia or any other country.

1.-25. (canceled)

26. A method of analysing an image for assessing a condition of an organ of a patient represented in the image, the method comprising the steps of:
selecting a spatial resolution for an inspection matrix comprising a plurality of inspection regions each delimiting a part of the image the spatial resolution for the inspection matrix is dependent on a size of the organ represented in the image or an age of the patient from whom the image of the organ is taken;
applying the inspection matrix to the image;
analysing the inspection regions to determine a condition thereof;
comparing the condition with a predetermined criterion;
identifying the inspection regions for which the condition is deemed to satisfy the predetermined criterion; and
providing a quantitative measure of an extent of the condition in the image based on a number of the identified inspection regions;
wherein the image is one of a plurality of images of the same organ of respective patients and the condition of that organ is assessed for each patient and wherein the step of selecting the spatial resolution for the inspection matrix is conducted for each patient and such that the spatial resolutions of the inspection matrices decreases substantially in linear proportion with an increase in a size of the organ of the patients.

27. The method according to claim 26, wherein the inspection matrix is a two-dimensional matrix.

28. The method according to claim 26, wherein the spatial resolution is selected so that each inspection region has a dimension being 1%, 2%, 3%, 4%, 5% or less of an overall dimension of the organ represented in the image.

29. The method according to claim 26 wherein the step of selecting the spatial resolution comprises selecting the spatial resolution such that, largely independent from age of a patient and size of the organ, the inspection matrices have substantially the same, or at least similar, number of inspection regions.

30. The method according to claim 26 wherein the image is a cross-sectional image and the dimension is a cross-sectional width dimension.

31. The method according to claim 26, wherein the condition is deemed to satisfy the predetermined criterion if the condition is prevalent over an area covering more than 10, 20, 30, 40, 50, 60, 70, 80 or 90% of the inspection region.

32. The method according to claim 26, wherein the condition is deemed not to satisfy the predetermined criterion if the condition is prevalent over an area covering more than 10, 20, 30, 40, 50, 60, 70, 80 or 90% of the inspection region.

33. The method according to claim 26, wherein the step of providing the quantitative measure comprises providing a proportion value of the number of identified inspection regions within the inspection matrix.

34. The method according to in claim 33, wherein providing the quantitative measure comprises counting a number of identified inspection regions and dividing this number by a total number of inspection regions in the inspection matrix.

35. The method according to claim 26, wherein the image is one of a computed tomography (CT) image, a radiograph image, and an MRI image.

36. The method according to claim 35, wherein the image comprises an inspiratory scan expiratory scan of the patient.

37. The method according to claim 26, wherein the criterion comprises the presence of a disease in the organ represented in the image.

38. The method according to claim 37, comprising establishing a marker of the disease if the quantitative measure exceeds a predetermined value.

39. The method according to claim 37, which is repeated at one or more distinct time intervals such that a change in the disease can be determined.

40. The method according to claim 26, which is repeated on a plurality of related images, wherein the plurality of related images comprises between one and five, six and fifteen, or approximately ten images of the organ.

41. The method according to claim 40, wherein the related images are equidistant two-dimensional slices taken through a three-dimensional image of the organ.

42. The method according to claim 26, wherein the image comprises a cross-sectional representation of the organ.

43. The method according to claim 26, wherein the image comprises a cross-sectional representation of a lung and wherein the condition is cystic fibrosis.

44. The method according to claim 26, wherein the image comprises an organ of a patient having an age of 10, 9, 8, 7, 6, 5, 4, 3, 2 or 1 years.

45. The method according to claim 26, wherein the steps of analysing the part of the image, comparing the condition thereof and identifying the inspection regions are all conducted autonomously by a computer system.

46. A method of analysing an image for assessing a condition of an organ of a patient represented in the image, the method comprising the steps of:
selecting a spatial resolution for an inspection matrix comprising a plurality of inspection regions each delimiting a part of the image the spatial resolution for the inspection matrix is dependent on a size of the organ represented in the image or an age of the patient from whom the image of the organ is taken;
applying the inspection matrix to the image;
analysing the inspection regions to determine a condition thereof;
comparing the condition with a predetermined criterion; and
identifying the inspection regions for which the condition is deemed to satisfy the predetermined criterion;
wherein the image is one of a plurality of images of the same organ of respective patients and the condition of that organ is assessed for each patient and wherein the step of selecting the spatial resolution for the inspection matrix is conducted for each patient and such that the spatial resolutions of the inspection matrices decreases substantially in linear proportion with an increase in a size of the organ of the patients.
